(19) United States
(12) Patent Application Publication
(10) Pub. No.: US 2026/0069872 A1
(43) Pub. Date: Mar. 12, 2026

(54) IMPLANTABLE CIRCUIT WITH STEP-DOWN CONVERTER FOR HIGH POWER INDUCTIVE CHARGE

(71) Applicant: Greatbatch Ltd., Clarence, NY (US)

(72) Inventors: Gabriel Amilcar Barbat, Ciudad de la Costa (UY); Agustín Villavedra, Montevideo (UY); Rocío Belén Curiel, Montevideo (UY)

(21) Appl. No.: 18/828,545

(22) Filed: Sep. 9, 2024

Related U.S. Application Data
(60) Provisional application No. 63/636,373, filed on Apr. 19, 2024.

(51) Int. Cl. A61N 1/378; A61N 1/02; A61N 1/372

(52) U.S. Cl. CPC A61N 1/3787; A61N 1/025; A61N 1/37217

(57) ABSTRACT

An implantable device includes a chargeable energy storage cell and a charging circuit. The charging circuit includes a secondary coil configured to receive charge energy inductively from a primary coil of an external device, an alternating current to direct current (AC-DC) converter circuit to configured to produce DC charge energy using the received charge energy, and a DC-DC step-down converter circuit configured to apply a stepped down DC charge energy to the energy storage element.

### CLAIM OF PRIORITY

This application claims the benefit of U.S. Provisional Application No. 63/636,373 filed on Apr. 19, 2024, which is hereby incorporated by reference in its entirety.

### TECHNICAL FIELD

Embodiments of the disclosure relate generally to body-implantable devices and more specifically to a charging circuit for a rechargeable energy storage element of a body-implantable device.

### BACKGROUND

Implantable devices include but are not limited to implantable pacemakers, implantable cardioverter defibrillators, implantable neurostimulators, and implantable heart pumps. The devices can be used to treat patients or subjects using electrical stimulation therapy or other therapy, or to aid a physician or caregiver in patient diagnosis through internal monitoring of a patient's condition. Some implantable medical devices can be diagnostic-only devices, such as implantable cardiac monitors, implantable loop recorders, and implantable heart failure monitors. Including a rechargeable battery in implantable devices extends the time the patient can use the device before the device needs to be removed or allows the device to provide higher energy therapy. Reducing the charge time improves patient quality of life, but it is challenging to transfer more power to reduce charge time while maintaining heating below safety limits and complying with electromagnetic compatibility standards.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, various embodiments discussed in the present document.

FIG. 1 is a block diagram of portions of an example of an implantable device.

FIG. 2 is a block diagram of portions of an example of a medical device system.

FIG. 3 is a circuit diagram of portions of an example of a charging circuit for an energy storage element of an implantable device.

FIG. 4 is a flow diagram of an example of a method of charging an energy storage element of an implantable device.

### DETAILED DESCRIPTION

Embodiments of the present disclosure are directed to controlling charging of an energy storage element used to power an implantable medical device. Implantable medical devices are typically battery-powered, and battery energy of the devices is consumed through delivery of electrical therapy and operation of circuits to record information sensed by the implantable device. Using a rechargeable battery extends the useful life of the implantable device before it needs to be removed and replaced.

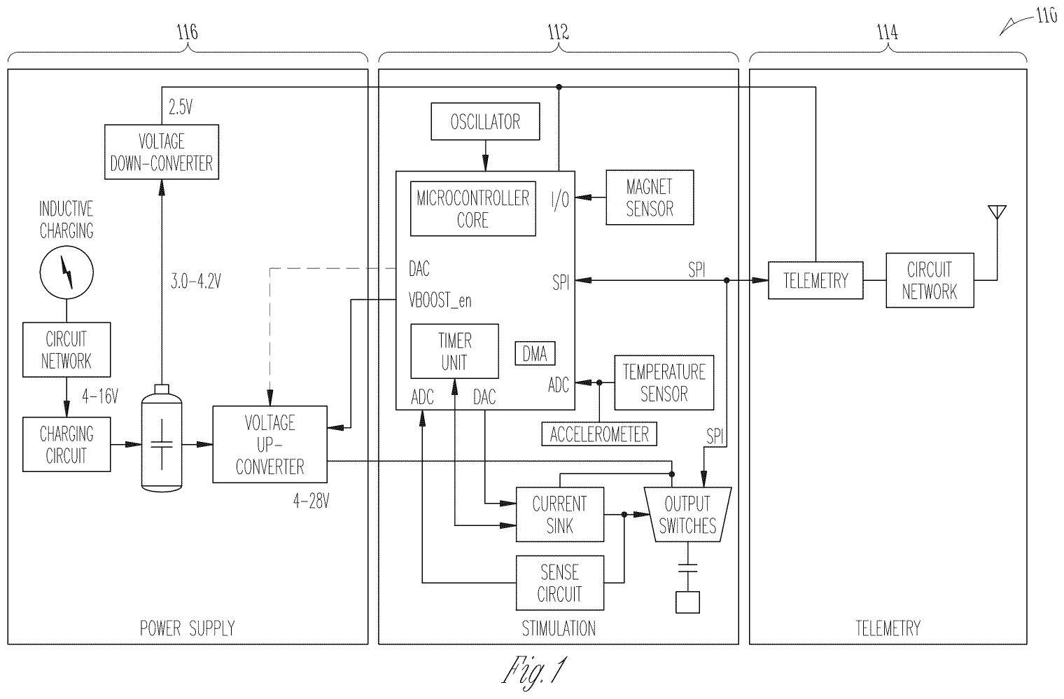

FIG. 1 is a block diagram of portions of an example of an implantable device 110. The implantable device 110 includes stimulation circuitry 112 that provides electrical stimulation therapy to a patient when connected to electrodes. The stimulation circuitry 112 may provide pacing stimulation therapy, cardioversion therapy, defibrillation therapy, or neurostimulation therapy. The stimulation circuitry 112 may sense physiologic electrical signals associated with the heart, spine or brain of the patient and deliver electrical stimulation therapy based on the sensed signals. The implantable device 110 includes communication circuitry 114 to communicate information with one or more external devices. The external devices may include a programmer that communicates one or more wireless signals with the implantable device 110, such as by using radio frequency (RF) or by one or more other telemetry methods. The external device can communicate information with the implantable device 110 to configure operation of the implantable device 110 by downloading operating parameters to the implantable device 110, and to upload data recorded by the implantable device 110 without removal of the implantable device 110. The implantable device 110 includes a microcontroller to control functions of the implantable device 110 such as timing of therapy delivery and communication of information. The implantable device 110 includes power supply circuitry 116 that can include a chargeable energy storage element (e.g., a rechargeable battery) and charging circuitry.

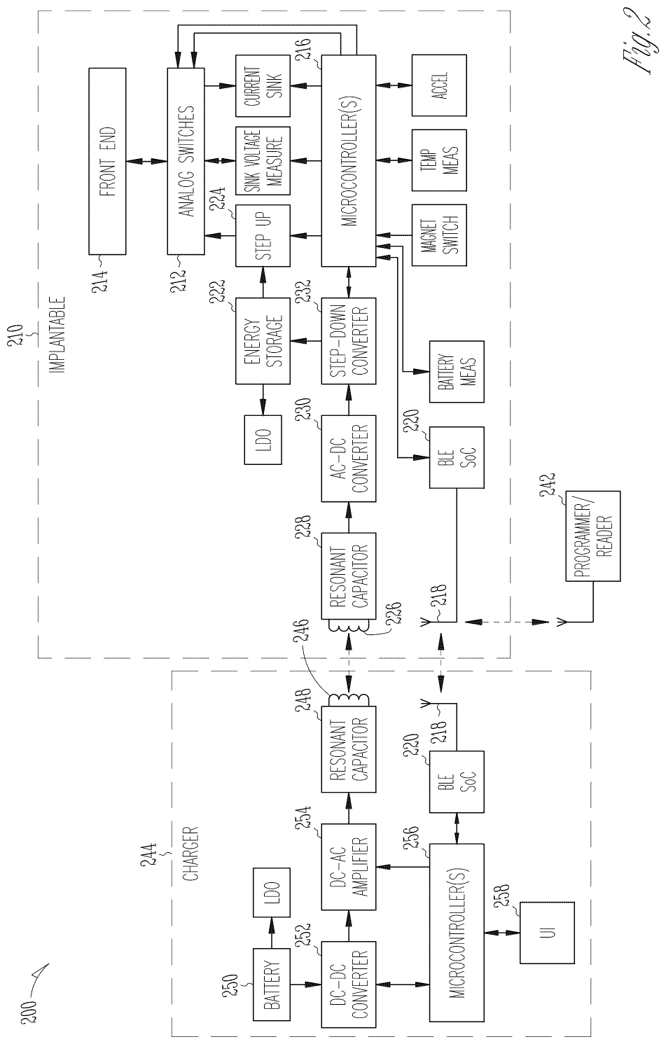

FIG. 2 is a block diagram of portions of an example of a medical device system. The system 200 includes an implantable device 210, an external programmer or reader device 242, and an external charger device 244. The implantable device 210 may be a pacemaker, cardioverter-defibrillators, neurostimulator, heart pump, or other implantable device. The implantable device 210 may deliver electrical stimulation therapy using analog switches 212 and front end 214. In some examples, the implantable device 210 is a diagnostic-only device that records sensed physiological signals using the analog switches 212 and front end 214. The implantable device 210 includes a controller such as one or more microcontrollers 216 to control the delivery of therapy and recording of information. A microcontroller 216 can include one or more of a microprocessor, an application specific integrated circuit (ASIC) programmable gate array (PGA), or other type of processor that executes instructions included in software or firmware.

The implantable device 210 includes a wireless communication link to communicate information to the programmer or reader device 242 and the charger device 244. In the example of FIG. 2, the wireless communication link includes an RF transceiver 220 (e.g., a Bluetooth Low Energy (BLE) system on chip (SoC)) and antenna 218. The microcontroller 216 communicates information with the programmer or reader device 242 and charger device 244 using the RF transceiver 220.

The implantable device 210 includes a chargeable energy storage element 222 to power the implantable device 210. The energy storage element may be a chargeable battery or a capacitor. The output of the energy storage element 222 is connected to a low dropout regulator circuit (LDO) to provide regulated supply to circuit blocks of the implantable device 210 such as microcontroller 216. The implantable device 210 includes a step-up converter circuit 224. The step-up converter circuit 224 may increase the voltage of energy from the energy storage element 222 to a voltage appropriate for the stimulation therapy provided by the implantable device 210. The implantable device 210 includes a charging circuit to charge the energy storage element 222. The energy storage element charging circuit includes a secondary coil 226, a resonant capacitor 228, an alternating current to direct current (AC-DC) converter circuit 230, and a step-down converter circuit 232.

The charger device 244 provides charge energy to the implantable device 210. In certain examples, the charger device 244 and the programmer or reader device 242 are combined in one external device. The charger device 244 includes a primary coil 246 and a resonant capacitor 248. In the example of FIG. 2, the charger device 244 is battery powered and includes a battery 250, DC-DC converter 252, and DC to AC amplifier 254. In variations, the charger device 244 is line powered. The charger device 244 includes one or more microcontrollers 256 and a communication link to communicate information to the implantable device 210. In the example of FIG. 2, the communication link includes an RF transceiver 220 and antenna 218. The charger device 244 also includes a user interface 258 (UI).

The secondary coil 226 of the implantable device 210 receives charge energy inductively from the primary coil 246 of the charger device 244. The implantable device 210 may include a metal housing. The secondary coil may be arranged outside the metal housing to receive the charge energy. The AC-DC converter circuit 230 converts the received AC charge energy to DC charge energy.

It is desirable to recharge the energy storage element 222 of the implantable device as quickly as possible. Charge time can be shortened by increasing the energy provided to the implantable device 210, but the charging must comply with safety levels of tissue heating by both the charger device 244 and the implantable device 210. There are standards that establish limits on the surface temperature of the surface of the implantable device 210, the surface of the charger device 244, and the specific absorption rate (SAR) due to the magnetic field generated by the charging operation. The operation of the charger device 244 and the implantable device 210 must also comply with electromagnetic compatibility (EMC) standards.

Within the limits of the standards, the charge time can be shortened by optimizing the efficiency of the charging. The maximum efficiency of the inductive charging link between the charger device 244 and the implantable device 210 is determined by the quality factor of the primary coil 246, the quality factor of the secondary coil 226, and the magnetic coupling of the coils. The maximum efficiency that can be obtained in the charging link of FIG. 2 depends on using appropriate resonant capacitors and having an appropriate load based on the secondary coil inductance.

In the case of a specific output charging energy, an appropriate load translates to an appropriate secondary voltage, which also relates to an appropriate AC-DC converter output voltage. In case of relatively high output power, the optimum output DC voltage of the AC-DC converter 230 for greater charging efficiency is often higher than the voltage of the energy storage element 222. The step-down converter circuit 232 provides a stepped down charge energy with a DC voltage appropriate to charge the energy storage element 222. For instance, the output of the AC-DC converter 230 may be 16 Volts, and the stepped down charge energy may be 4.2 Volts. Using the step-down converter circuit 232 in the charging circuit of the implantable device 210 allows charge energy to be transferred at the higher voltage corresponding to higher efficiency and then stepped down to the voltage level appropriate to charge the energy storage element 222.

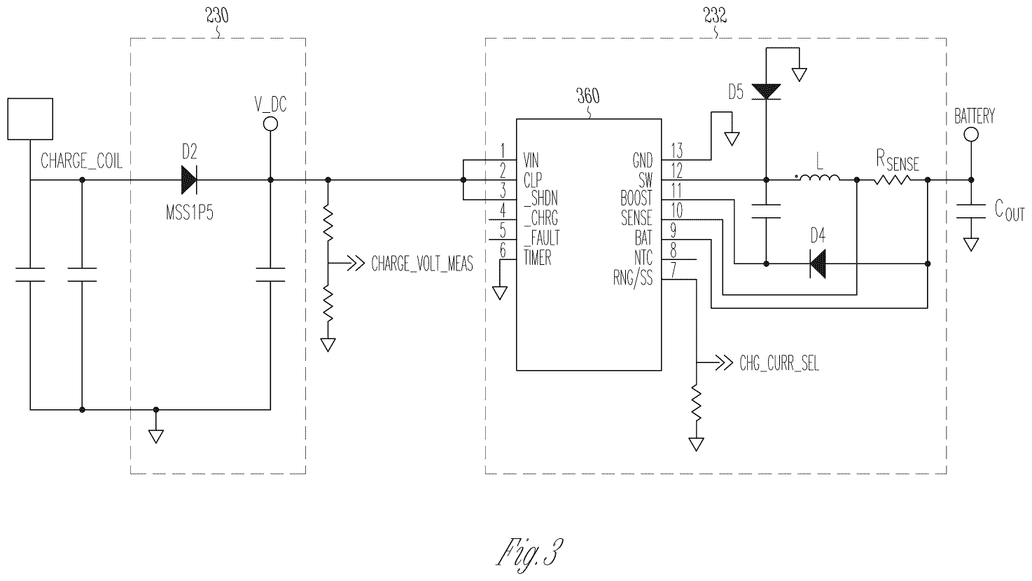

FIG. 3 is a circuit diagram of examples of the AC-DC converter circuit 230 and the DC-DC step-down converter circuit 232. The DC-DC step-down converter circuit 232 includes a step-down charge integrated circuit (IC). The DC-DC step-down converter circuit 232 is a switching converter circuit and the step-down charge IC 360 controls the switching to charge and discharge the inductor L. The step-down charge IC 360 monitors the charge current applied to the energy storage element 222 using sense resistor R_{SENSE} and it performs the control for constant current and constant voltage charge phases. The step-down charge IC 360 can be programmed by the microcontroller 216 in order to set the constant current phase. For relatively high magnetic coupling between the primary coil 246 and the secondary coil 226, the charge current can be set to the maximum charge current level specified for the energy storage element 222 and the power transmitted by the charger device 244 may be reduced for maximizing efficiency. For instance, the charge current can be set to as high as 500 milliamps (500 mA), while the maximum voltage of the energy storage element is 4.2V in constant voltage phase. For relatively low magnetic couplings, in which cases the output current cannot reach the maximum allowed by the energy storage element 222, the charger device 244 may transmit maximum power to maximize charge current and finally reach the maximum charge voltage but after a longer time.

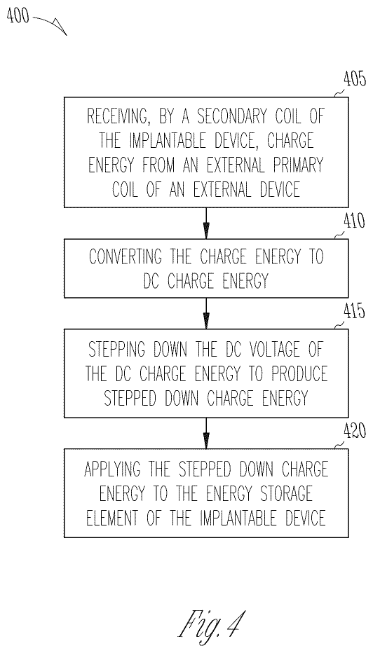

FIG. 4 is a flow diagram of an example of a method 400 of charging an energy storage element of an implantable device (e.g., implantable device 210 in FIG. 2). At block 405, the secondary coil 226 of the implantable device 210 receives charge energy from the external primary coil 246 of the external charger device 244. The received charge energy may be AC charge energy having a voltage amplitude corresponding to the highest efficiency energy transfer using inductive coupling between the primary and second coils.

At block 410, the charge energy received by the secondary coil 226 is converted to DC charge energy. In some examples, the received charge energy is converted to DC charge energy using an AC-DC rectifier circuit. At block 415, the DC voltage of the DC charge energy is stepped down using a DC-DC step-down converter to produce stepped down charge energy. The stepped down charge energy has a DC voltage corresponding to the charging voltage appropriate for charging the energy storage element 222. At block 420, the stepped down DC charge energy is applied to the energy storage element 222 to charge the energy storage element 222.

The controller of the implantable device 210 (e.g., microcontroller 216) is configured (e.g., through software or firmware) to set the charge current level of the stepped down charge energy used to charge the energy storage element 222. In some examples, the controller is configured to measure the charge current applied to the battery and the voltage of the battery. For instance, the controller may monitor the voltage across the sense resistor in FIG. 3 to determine the charge current and measure the voltage at the output capacitor C_{OUT}. The controller may also monitor the output of the AC-DC converter circuit 230 using a resistor divider (CHARGE_VOLT_MEASURE). The controller can set the current output of the step-down charge IC 360 by setting the voltage on the charge current select input (CHG_CURR_SEL) of the step-down converter circuit 232.

In some examples, the controller of the implantable device 210 sends charge information to the controller of the external charger device during charging of the energy storage element 222 (e.g., using the wireless communication link in FIG. 2). The charge information can include measurements of one or more of the charge current, the voltage of the energy storage element 222, the voltage of the output of the AC-DC converter, and other feedback from the implantable device regarding received charging energy. The controller of the charger device 244 can adjust the energy level transmitted based on the charge information. The controller of the implantable device 210 sets the charge current level to the charge energy received from the charger device 244. The battery charge information can include the recharge level of the battery 222. The controller of the implantable device 210 may also send status of the electrical stimulation therapy provided by the implantable device 210 to the charger device 244 during charging.

In some examples, the controller of the charger device 244 sends a request for charging information to the controller of the implantable device 210 during charging. In response to the request, the controller of the implantable device 210 returns values for one or more of the charge current, the voltage of the energy storage element, and the voltage of the output of the AC-DC converter. The controller of the charger device 244 adjusts the charge energy provided by the primary coil according to the received information. For instance, if the value of charge current received from the implantable device 210 is less than a predetermined threshold current, the controller of the charger device 244 may increase the charge energy. If the voltage of the output of the AC-DC converter 230 is less than a predetermined threshold voltage, the controller of the charger device 244 may increase the charge energy. If the voltage of the output of the AC-DC converter 230 is more than a predetermined threshold voltage, the controller of the charger device 244 may decrease the charge energy. In some examples, the implantable microcontroller 216 processes the measurements and determines if the charger device 244 should attempt to increase or decrease the transmitted charge energy and provides the feedback to the charger device 244.

Being able to adjust the voltage of the output of the AC-DC converter 230 to a predetermined target voltage allows the charger device 244 to adjust the secondary coil voltage to achieves an optimum efficiency point of the charging power transfer. This allows a high-power output of charge energy while keeping any heating of tissue within acceptable limits. Because efficiency is optimized by the charging system, the size of the secondary coil of the implantable device can be minimized to achieve the power transfer requirements. For low levels of magnetic coupling of the coils (as in deeper implants) the charge current can be reduced while maximizing efficiency of the charging power transfer.

### ADDITIONAL DESCRIPTION AND EXAMPLES

Example 1 includes subject matter (such as an implantable device) comprising a chargeable energy storage element and a charging circuit. The charging circuit includes a secondary coil configured to receive charge energy inductively from a primary coil of an external device, an alternating current to direct current (AC-DC) converter circuit configured to produce DC charge energy using the received charge energy, and a DC-DC step-down converter circuit configured to apply a stepped down DC charge energy to the energy storage element.

In Example 2, the subject matter of Example 1 optionally includes a controller configured to set a charge current level of the stepped down DC charge energy.

In Example 3, the subject matter of one or both of Examples 1 and 2 optionally includes a controller configured to measure charge current applied to the energy storage element and set a charge current level of the stepped down DC charge energy according to the measured charge current.

In Example 4, the subject matter of one or any combination of Examples 1-3 optionally includes a communication circuit configured to communicate information wirelessly with the external device, and a controller configured to send information related to received charge energy to the external device.

In Example 5, the subject matter of Example 4 optionally includes a controller configured to measure at least one of an output voltage of the AC-DC converter, a charge current, and voltage of the energy storage cell.

In Example 6, the subject matter of one or any combination of Examples 1-5 optionally includes a communication circuit configured to communicate information wirelessly with the external device, and a controller configured to send a measurement of voltage of the energy storage element to the external device.

In Example 7, the subject matter of one or any combination of Examples 1-6 optionally includes a therapy circuit configured to deliver electrical stimulation therapy when connected to electrodes, a communication circuit configured to communicate information wirelessly with the external device, and a controller configured to send status of the electrical stimulation therapy to the external device.

Example 8 includes subject matter (such as a method of charging an energy storage element of an implantable device) or can optionally be combined with one or any combination of Examples 1-7 to include such subject matter, comprising receiving, by a secondary coil of the implantable device, charge energy from an external primary coil of an external device; converting the charge energy to direct current (DC) charge energy; stepping down a DC voltage of the DC charge energy to produce stepped down charge energy; and applying the stepped down charge energy to the energy storage element of the implantable device.

In Example 9, the subject matter of Example 8 optionally includes measuring a voltage of the DC charge energy using the implantable device, sending a voltage measurement of the DC charge energy from the implantable device to the external device, and adjusting the charge energy of the external device according to the voltage measurement.

In Example 10, the subject matter of one or both of Example 8 and 9 optionally includes measuring charging current applied to the energy storage cell using the implantable device; and adjusting, by a controller of the implantable device, the charging current to a target charging current value.

In Example 11, the subject matter of one or any combination of Examples 8-10 optionally includes sending a measurement related to received charge energy from the implantable device to the external device; and adjusting, by the external device, the charge energy according to the measurement.

In Example 12, the subject matter of Example 11 optionally includes determining, by the implantable device, a measurement of at least one of an output voltage of the AC-DC converter, a charge current, and voltage of the energy storage cell; and sending the measurement to the external device.

In Example 13, the subject matter of one or any combination of Examples 8-12 optionally includes sending energy storage cell charge information from the implantable device to the external device during charging of the energy storage cell.

In Example 14, the subject matter of one or any combination of Examples 8-13 optionally includes sending status of an electrical stimulation therapy provided by the implantable device to the external device during charging of the energy storage cell.

Example 15 includes subject matter (such as a medical device system) or can optionally be combined with one or any combination of Examples 1-14 to include such subject matter, comprising an implantable device including a chargeable energy storage cell and an external device including a primary charging coil. The implantable device further includes a charging circuit that includes a secondary coil configured to receive charge energy inductively from the primary coil of the external device, an alternating current to direct current (AC-DC) converter circuit configured to produce DC charge energy using the received charge energy, and a DC-DC step-down converter circuit configured to apply a stepped down charge energy to the energy storage cell.

In Example 16, the subject matter of Example 15 optionally includes the implantable device including a first communication circuit configured to communicate information wirelessly with the external device, and an implantable device controller configured to send information related to charging of the energy storage cell to the external device using the first communication circuit. The external device optionally includes a second communication circuit configured to communicate information wirelessly with the implantable device, and an external device controller configured to receive the information related to charging of the energy storage cell and adjust the charge energy of the primary coil according to the information.

In Example 17, the subject matter of Example 16 optionally includes an implantable device controller configured to send a measurement of one or both of charging current and voltage of the AC-DC converter to the external device using the first communication circuit; and an external device controller is configured to adjust the charge energy of the primary coil according to the measurement.

In Example 18, the subject matter of one or both of Examples 16 and 17 optionally includes an implantable device controller configured to send a measurement of voltage of the energy storage element to the external device.

In Example 19, the subject matter of one or any combination of Examples 15-18 optionally includes the implantable device including an implantable device controller configured to monitor charge current and set a charge current level of the stepped down charge energy.

In Example 20, the subject matter of Example 19 optionally includes the implantable device controller configured to change the charge current level to minimize a charge time of the energy storage cell.

These non-limiting Examples can be combined in any permutation or combination. The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F. R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. An implantable device comprising:
a chargeable energy storage element; and
a charging circuit including:
a secondary coil configured to receive charge energy inductively from a primary coil of an external device;
an alternating current to direct current (AC-DC) converter circuit configured to produce DC charge energy using the received charge energy; and
a DC-DC step-down converter circuit configured to apply a stepped down DC charge energy to the energy storage element.

2. The implantable device of claim 1, including a controller configured to set a charge current level of the stepped down DC charge energy.

3. The implantable device of claim 1, including a controller configured to:
measure charge current applied to the energy storage element; and
set a charge current level of the stepped down DC charge energy according to the measured charge current.

4. The implantable device of claim 1, including:
a communication circuit configured to communicate information wirelessly with the external device; and
a controller configured to send information related to received charge energy to the external device.

5. The implantable device of claim 4,
wherein the controller is configured to measure at least one of an output voltage of the AC-DC converter, a charge current, and voltage of the energy storage cell.

6. The implantable device of claim 1, including:
a communication circuit configured to communicate information wirelessly with the external device; and
a controller configured to send a measurement of voltage of the energy storage element to the external device.

7. The implantable device of claim 1, including:
a therapy circuit configured to deliver electrical stimulation therapy when connected to electrodes;
a communication circuit configured to communicate information wirelessly with the external device; and
a controller configured to send status of the electrical stimulation therapy to the external device.

8. A method of charging an energy storage element of an implantable device, the method comprising:
receiving, by a secondary coil of the implantable device, charge energy from an external primary coil of an external device;
converting the charge energy to direct current (DC) charge energy;
stepping down a DC voltage of the DC charge energy to produce stepped down charge energy; and
applying the stepped down charge energy to the energy storage element of the implantable device.

9. The method of claim 8, including:
measuring a voltage of the DC charge energy using the implantable device;
sending a voltage measurement of the DC charge energy from the implantable device to the external device; and
adjusting the charge energy of the external device according to the voltage measurement.

10. The method of claim 8, including:
measuring charging current applied to the energy storage cell using the implantable device; and
adjusting, by a controller of the implantable device, the charging current to a target charging current value.

11. The method of claim 8, including:
sending a measurement related to received charge energy from the implantable device to the external device; and
adjusting, by the external device, the charge energy according to the measurement.

12. The method of claim 11, including:
determining, by the implantable device, a measurement of at least one of an output voltage of the AC-DC converter, a charge current, and voltage of the energy storage cell; and
sending the measurement to the external device.

13. The method of claim 8, including:
sending energy storage cell charge information from the implantable device to the external device during charging of the energy storage cell.

14. The method of claim 8, including:
sending status of an electrical stimulation therapy provided by the implantable device to the external device during charging of the energy storage cell.

15. A medical device system, the system comprising:
an implantable device including a chargeable energy storage cell; and
an external device including a primary charging coil; and
wherein the implantable device further includes a charging circuit including:
a secondary coil configured to receive charge energy inductively from the primary coil of the external device;
an alternating current to direct current (AC-DC) converter circuit configured to produce DC charge energy using the received charge energy; and
a DC-DC step-down converter circuit configured to apply a stepped down charge energy to the energy storage cell.

16. The system of claim 15,
wherein the implantable device further includes:
a first communication circuit configured to communicate information wirelessly with the external device; and
an implantable device controller configured to send information related to charging of the energy storage cell to the external device using the first communication circuit;
wherein the external device further includes:
a second communication circuit configured to communicate information wirelessly with the implantable device; and
an external device controller configured to receive the information related to charging of the energy storage cell and adjust the charge energy of the primary coil according to the information.

17. The system of claim 16,
wherein the implantable device controller is configured to send a measurement of one or both of charging current and voltage of the AC-DC converter to the external device using the first communication circuit; and
wherein the external device controller is configured to adjust the charge energy of the primary coil according to the measurement.

18. The system of claim 16, wherein the implantable device controller is configured to send a measurement of voltage of the energy storage element to the external device.

19. The system of claim 15, wherein the implantable device further includes an implantable device controller configured to monitor charge current and set a charge current level of the stepped down charge energy.

20. The system of claim 19, wherein the implantable device controller is configured to change the charge current level to minimize a charge time of the energy storage cell.
